(19) United States
(12) Patent Application Publication
(10) Pub. No.: US 2022/0384049 A1
(43) Pub. Date: Dec. 1, 2022

(54) ENSEMBLE GENERATIVE ADVERSARIAL NETWORK BASED SIMULATION OF CARDIOVASCULAR DISEASE SPECIFIC BIOMEDICAL SIGNALS

(71) Applicant: Tata Consultancy Services Limited, Mumbai (IN)

(72) Inventors: ROHAN BANERJEE, Kolkata (IN); AVIK GHOSE, Kolkata (IN)

(73) Assignee: Tata Consultancy Services Limited, Mumbai (IN)

(21) Appl. No.: 17/472,315

(22) Filed: Sep. 10, 2021

(30) Foreign Application Priority Data
May 3, 2021 (IN) 202121020228

(51) Int. Cl. G16H 50/50; G16H 50/70; G06N 3/08

(52) U.S. Cl. CPC G16H 50/50; G16H 50/70; G06N 3/08

(57) ABSTRACT

Computer-aided diagnosis algorithms require a large volume of training data. The existing methods for simulating artificial biomedical signals are mostly based on physics driven mathematical models that require too many assumptions, making them challenging to simulate on a large scale. Alternatively, conventional deep learning-based approaches are pure data driven and hence, do not have physiological interpretation. The present disclosure provides a method that effectively combines both physiological domain knowledge and deep learning to enable simulation of realistic cardiovascular disease specific biomedical signals. An ensemble Generative Adversarial Network (GAN) including a Long Short-Term Memory GAN (LSTM-GAN) configured to generate a Heart Rate Variability (HRV) pattern associated with the cardiovascular disease condition and a Deep Convolutional GAN (DCGAN) configured to create a morphology of a representative cardiac cycle is provided. A complete waveform is simulated by combining an output from each GAN.

### PRIORITY CLAIM

This U.S. patent application claims priority under 35 U.S.C. § 119 to: Indian Patent Application No. 202121020228, filed on 3 May, 2021. The entire contents of the aforementioned application are incorporated herein by reference.

### TECHNICAL FIELD

The disclosure herein generally relates to the field of simulation of cardiovascular disease specific biomedical signals, and, more particularly, to systems and methods for simulation of cardiovascular disease specific biomedical signals using an ensemble Generative Adversarial Network (GAN).

### BACKGROUND

Electrocardiogram (ECG) is a clinical test to record the electrophysiological activities of the heart. The recorded data is interpreted by doctors for non-invasive diagnosis of cardiovascular diseases. However, it is practically impossible to manually analyze the large volume of ECG data generated each day in a hospital. An automatic diagnosis from the digitally recorded ECG is possible using artificial intelligence, machine learning, and deep learning techniques. A supervised learning-based cardiac diagnosis algorithm typically requires a large volume of annotated data as a prerequisite to creating the training model. Recording of large-scale patient data is time consuming and often challenging due to privacy issues and associated risks in case of infectious diseases. This is addressed in data science by generating synthetic patient data.

Although normal ECG samples are substantially available in various open-access databases, the quantity of abnormal recordings corresponding to different heart diseases is often inadequate to train a machine learning or deep learning classifier. The existing methods for simulating artificial ECG signals are mostly based on physics driven mathematical model. However, they require too many assumptions, for instance, parameters of the heart, thereby making them challenging to simulate on a large scale. On the other hand, deep learning-based approaches are also popular in literature. But these are pure data driven approach and hence, do not have physiological interpretation, for want of real data.

### SUMMARY

Embodiments of the present disclosure present technological improvements as solutions to one or more of the above-mentioned technical problems recognized by the inventors in conventional systems.

In an aspect, there is provided a processor implemented method comprising the steps of: receiving as input, via one or more hardware processors, (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles; training an ensemble Generative Adversarial Network (GAN) comprising a pair of GANs, via the one or more hardware processors, using the received input, wherein the pair of GANs includes (i) a Long Short-Term Memory GAN (LSTM-GAN) configured to generate a Heart Rate Variability (HRV) pattern associated with the cardiovascular disease condition and (ii) a Deep Convolutional GAN (DCGAN) configured to create a morphology of a representative cardiac cycle from the plurality of complete cardiac cycles, and wherein each GAN in the pair of GANs includes a generator and a discriminator; and simulating, via the one or more hardware processors, a time series data representing the biomedical signal by combining an output from each GAN in the pair of GANs.

In another aspect, there is provided a system comprising: one or more hardware processors; one or more communication interfaces; one or more data storage devices operatively coupled to the one or more hardware processors and configured to store instructions configured for execution by the one or more hardware processors to: receive as input (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles; train an ensemble Generative Adversarial Network (GAN) comprising a pair of GANs, using the received input, wherein the pair of GANs includes (i) a Long Short-Term Memory GAN (LSTM-GAN) configured to generate a Heart Rate Variability (HRV) pattern associated with the cardiovascular disease condition and (ii) a Deep Convolutional GAN (DCGAN) configured to create a morphology of a representative cardiac cycle from the plurality of complete cardiac cycles, and wherein each GAN in the pair of GANs includes a generator and a discriminator; and simulate a time series data representing the biomedical signal by combining an output from each GAN in the pair of GANs; and the ensemble Generative Adversarial Network (GAN) comprising the pair of GANs.

In yet another aspect, there is provided a computer program product comprising a non-transitory computer readable medium having a computer readable program embodied therein, wherein the computer readable program, when executed on a computing device, causes the computing device to: receive as input (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles; train an ensemble Generative Adversarial Network (GAN) comprising a pair of GANs, using the received input, wherein the pair of GANs includes (i) a Long Short-Term Memory GAN (LSTM-GAN) configured to generate a Heart Rate Variability (HRV) pattern associated with the cardiovascular disease condition and (ii) a Deep Convolutional GAN (DCGAN) configured to create a morphology of a representative cardiac cycle from the plurality of complete cardiac cycles, and wherein each GAN in the pair of GANs includes a generator and a discriminator; and simulate, a time series data representing the biomedical signal by combining an output from each GAN in the pair of GANs.

In accordance with an embodiment of the present disclosure, the one or more hardware processors are configured to train the ensemble GAN by performing, for each training epoch, the steps of: generating an R-R interval time series, by the generator of the LSTM-GAN, using the first set of real numbers by mapping the first set of real numbers to a time series and classifying the generated R-R interval time-series as belonging to the cardiovascular disease condition or not, by the discriminator of the LSTM-GAN based on R-R interval distances computed using the second set of reference training data; and generating the representative cardiac cycle specific to the cardiovascular disease condition, by the generator of the DCGAN, using the first set of real numbers and classifying the generated representative cardiac cycle as belonging to the cardiovascular disease condition or not, by the discriminator of the DCGAN based on the cardiac cycle computed using the second set of reference training data, wherein the step of training culminates after a first predefined number of training epochs or when the generated R-R interval time series and the generated representative cardiac cycle is within a predefined threshold when compared with the second set of reference training data.

In accordance with an embodiment of the present disclosure, the one or more hardware processors are configured to periodically validate the step of training by simulating the time series data representing the biomedical signal by combining an output from each GAN in the pair of GANs, repeatedly, after every second predefined number of training epochs, where in the second predefined number of training epochs is less than the first predefined number of training epochs.

In accordance with an embodiment of the present disclosure, the one or more hardware processors are configured to simulate the time series data representing the biomedical signal by modifying length of the representative cardiac cycle generated by the DCGAN according to the R-R interval distances generated by the LSTMGAN using cubic spline interpolation and appending the representative cardiac cycles on a time axis.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates an exemplary block diagram of a system for simulation of cardiovascular disease specific biomedical signals using an ensemble Generative Adversarial Network (GAN), in accordance with some embodiments of the present disclosure.

FIG. 2 illustrates an exemplary architecture of the ensemble GAN for simulation of cardiovascular disease specific biomedical signals, in accordance with some embodiments of the present disclosure.

FIG. 3A and FIG. 3B illustrate Electrocardiogram (ECG) patterns of a normal subject and an Atrial Fibrillation (AF) patient, as known in the art.

FIG. 4 illustrates an exemplary flow diagram of a computer implemented method for simulation of cardiovascular disease specific biomedical signals using the ensemble GAN, in accordance with some embodiments of the present disclosure.

FIG. 5 illustrates sample ECG waveforms generated, in accordance with some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Artificial intelligence (AI), machine learning and deep learning techniques are used in healthcare applications for various diagnosis and simulation activities. The success of such algorithms heavily depends upon the size and the diversity of data used for training. However, recording of large-scale patient data is often difficult and time consuming due to underlying security and privacy issues as well as risks involved in case of infectious diseases.

The existing methods for simulating artificial Electrocardiogram (ECG) signals are mostly based on physics driven mathematical model. However, they require too many assumptions, for instance parameters of the heart, making them challenging to simulate on a large scale. On the other hand, deep learning-based approaches are purely data driven and hence, do not have physiological interpretation.

The deep learning approaches try to simulate artificial data by learning the distribution from a real-world training dataset. The Generative Adversarial Network (GAN) is a popular example of such generative modeling which is extensively used for generating realistic images and time-series data. Zhu et al. proposed a GAN architecture using the MIT-BIH arrhythmia database in Scientific reports 2019. However, the utility of the generated ECG data was not qualitatively evaluated. The GAN proposed by Hatamian et al. in ICASSP 2020 generates the spectrogram of ECG corresponding to Atrial Fibrillation (AF), but not the waveforms. Abnormal ECG waveforms contain various anomalous patterns owing to their underlying conditions, making them difficult to simulate using pure statistical approaches

The present disclosure effectively combines both physiological domain knowledge and deep learning to artificially simulate realistic cardiovascular disease specific biomedical signals. Atrial fibrillation (AF), a common type of arrhythmia has been used as an exemplary use case in the description. Although the method and system provided in the present disclosure may be applied to any biomedical signal such as ECG, Photoplethysmogram (PPG) or Phonocardiogram (PCG), the description below is directed to a non-limited example of periodic time series data (signal) such as ECG. Accordingly, ECG signal and biomedical signals may be used interchangeably. So also, cardiovascular disease and AF may be used interchangeably.

In the context of the present disclosure, the expressions ‘time series data’, ‘signal’ and ‘waveform’ may be used interchangeably. Accordingly, multiple cycles form a waveform.

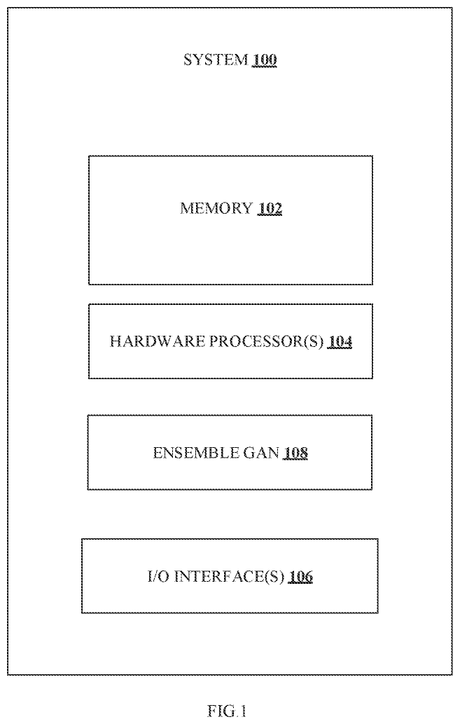

Referring now to the drawings, and more particularly to FIG. 1 through FIG. 5, where similar reference characters denote corresponding features consistently throughout the figures, there are shown preferred embodiments and these embodiments are described in the context of the following exemplary system and/or method.

FIG. 1 illustrates an exemplary block diagram of a system 100 for simulation of cardiovascular disease specific biomedical signals using an ensemble Generative Adversarial Network (GAN), in accordance with some embodiments of the present disclosure. In an embodiment, the system 100 includes one or more hardware processors 104, communication interface (s) or input/output (I/O) interface(s) 106, one or more data storage devices or memory 102 operatively coupled to the one or more hardware processors 104, and the ensemble GAN 108 described later in the description. The one or more hardware processors 104 can be implemented as one or more microprocessors, microcomputers, microcontrollers, digital signal processors, central processing units, state machines, graphics controllers, logic circuitries, and/or any devices that manipulate signals based on operational instructions. Among other capabilities, the processor(s) are configured to fetch and execute computer-readable instructions stored in the memory. In the context of the present disclosure, the expressions ‘processors’ and ‘hardware processors’ may be used interchangeably. In an embodiment, the system 100 can be implemented in a variety of computing systems, such as laptop computers, notebooks, hand-held devices, workstations, mainframe computers, servers, a network cloud and the like.

The communication interface (s) 106 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite. In an embodiment, the I/O interface(s) can include one or more ports for connecting a number of devices to one another or to another server.

The memory 102 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. In an embodiment, one or more modules (not shown) of the system 100 can be stored in the memory 102.

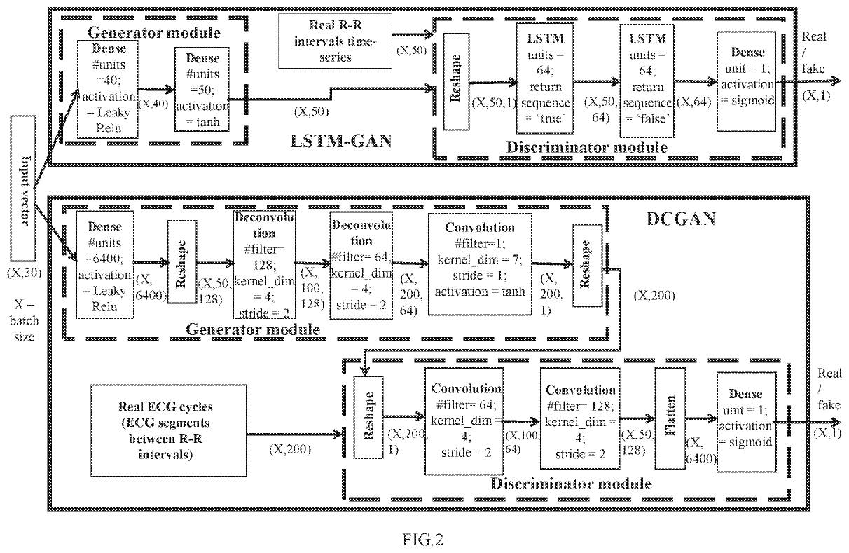

FIG. 2 illustrates an exemplary architecture of the ensemble GAN for simulation of cardiovascular disease specific biomedical signals, in accordance with some embodiments of the present disclosure. In accordance with the present disclosure the ensemble GAN comprises a pair of GANs viz., (i) a Long Short-Term Memory GAN (LSTM-GAN) configured to generate a Heart Rate Variability (HRV) pattern associated with the cardiovascular disease condition and (ii) a Deep Convolutional GAN (DCGAN) configured to create a morphology of a representative cardiac cycle from the plurality of complete cardiac cycles, wherein each GAN in the pair of GANs includes a generator and a discriminator.

The generator G takes an N-dimensional latent vector z as input that follows a Gaussian distribution and maps it to a generated data as its output, G(z). The discriminator D outputs D(G(z)), a probability to predict whether the generated data is real, or fake based on a training set of real data, x. The generator and the discriminator reach a convergence state via a zero-sum game. The objective function of a GAN is expressed in terms of a min-max optimization process as in equation (1) below.

$${min}_{G} {max}_{D} V ( D , G ) = {E}_{x \sim P ( x )} [ log D ( x ) ] + {E}_{z \sim P ( z )} [ log ( 1 - D ( G ( z ) ) ) ] \qquad ( 1 )$$

D tries to maximize the probability to correctly classify real and fake data, and G tries to minimize the probability that D may predict its output as fake. Under an optimum state, distribution of the fake data becomes equivalent to the real data, and the discriminator classifies them at probability of 0.5.

An exemplary embodiment of the ensemble GAN of the present disclosure as shown in FIG. 2 is described herein below. A batch of 30-dimensional latent vectors randomly sampled from a standard normal distribution is fed to both generators as input during training. A set of annotated AF recordings are obtained from the PhysioNet Challenge 2017 database to form the real data.

The LSTM-GAN for generating R-R interval distances: Irregular HRV is a known clinical marker for AF. An LSTM is a deep learning architecture that has its internal memory for sequential modeling of time-series data in terms of a hidden vector. It can effectively learn the desired pattern from a very long sequence due to a unique cell structure, that enables to delete less important information from memory. The R-R interval distances extracted from an ECG data may be represented as a vector rrₜ of k real numbers. Here, rrₜ=[rr₁,rr₂, . . . rrₖ], where rrᵢ=rᵢ₊₁−rᵢ, and rᵢ is the location of the i^{th}R peak in the ECG signal on a time axis. The real R-R interval distances computed from the PhysioNet database for training the discriminator. The number of points in the vectors varies due to different lengths of the recordings in the database. Considering the median duration, length of an R-R intervals vector is set to 50 in the network. The shorter recordings are repeated and merged accordingly, whereas the longer recordings are broken into multiple partially overlapping segments to increase the instances of real data.

The generator and the discriminator are both designed using non-linear neural networks. Input to the generator is a set of real numbers having length of 30, randomly picked from a unit Gaussian distribution. The generator contains two dense layers (a set of fully connected neurons) that takes the input vector corresponding to the set of real numbers and maps to a vector which is equal to the number of neurons in the dense layers. The two dense layers of the generator have 40 and 50 neurons respectively, hence the output of the generator has a length of 50 which is the desired length of the generated R-R intervals by the architecture. Leaky Rectified Linear Unit (Leaky Relu) with negative slope coefficient=0.2 and hyperbolic tangent (tanh) functions are used for non-linear activation of the units in the two layers. The discriminator takes both the generator data and a reference R-R intervals time series as input and classifies whether the generated data is real or not. The inputs are required to be reshaped before applying them to the LSTM layers. The classifier in the discriminator contains a pair of LSTM layers (each having 64 units), that generate a hidden vector that maps the temporal relation of the input and then it is sent to a dense layer having a single neuron with sigmoid activation function for binary classification.

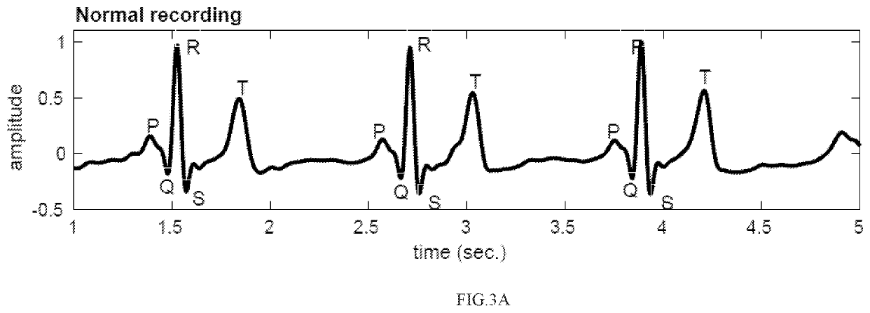

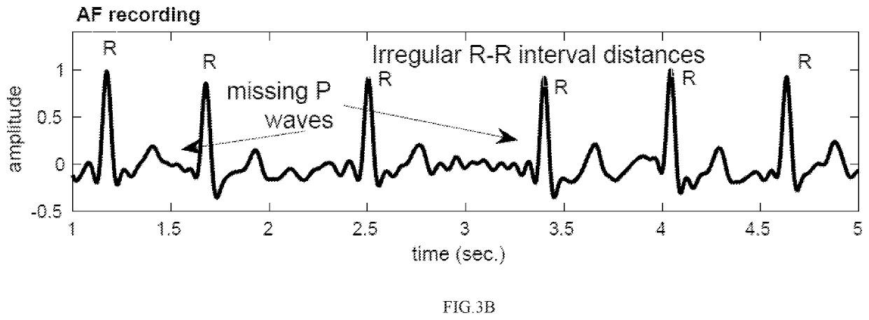

The DCGAN for generating signal morphology between two adjacent R peaks: Normal atrial activities of the heart become awry due to AF. This is reflected in the ECG morphology in terms of missing P waves or presence of abnormal fibrillatory waves before a QRS complex (Refer FIG. 3B described below). The DCGAN structure to generate ECG cycles incorporating such unique morphology. As shown in equation (2) below, an ECG time-series, ecgₜ may be represented as a vector of m real numbers, containing a set of landmark points as the R peaks of known locations.

ecgₜ=[ecg₁,ecg₂, . . . ecgᵣ_{₁},ecgᵣ_{₂}, . . . ecgₘ [symbol] (2)

Here rᵢ indicates the time location of the i^{th}R peak in the ECG signal and ecgᵣ_{ᵢ} is the corresponding amplitude. An ECG cycle is defined, in the present disclosure, as the segment between two adjacent R peaks. The p^{th} cycle is extracted as given in equation (3) below.

cycleₚ=[ecg₍ᵣ_{ₚ₎},ecg₍ᵣ_{ₚ₊₁₎},ecg₍ᵣ_{ₚ₊₂}), . . . ecg₍ᵣ_{ₚ₊ₚ}⁻¹⁾ [symbol] (3)

In the DCGAN, the lower dimensional latent vector is converted to a desired space of realistic generated data based on a series of convolution and transposed-convolution operations in the discriminator and the generator through a set of filters (kernel). The real ECG cycles are extracted from the PhysioNet Challenge database. The signals are sampled at 300 Hz. Since AF causes a heart rate faster than the normal range, length of an ECG cycle to be generated by the DCGAN model is fixed to 200 (≈667 ms long, instantaneous heart rate=90 bpm) in the architecture of the present disclosure. Duration of every real ECG cycle is modified accordingly using cubic spline interpolation technique before applying to the discriminator.

The generator takes the input vector of length 30 as input and first applies it to a dense layer having 6400 units to map it to a higher dimensional space. Subsequently, the output is applied to a pair of de-convolutional layers for extraction of relevant features. The output of the second deconvolutional layer is applied to another convolutional layer having a single filter and reshaped accordingly to get a required length, 150 of the generated cycle. The discriminator classifies whether the generated cycle is real or fake by comparing with a set of real cycles. After reshaping the classifier input for applying for convolution operation, the discriminator uses a pair of 1-Dimensional convolution layers with associated batch normalization and Leaky Relu activation layers. The convolution layers contain 64 and 128 filters respectively for relevant feature extraction. The output of the convolution operation is multi-dimensional. Hence, the output of the final convolutional layer is flattened to get a 1-Dimensional vector which is applied to a single neuron dense layer with sigmoid activation function for binary classification. To mitigate the chance of over-fitting, 30% dropout is applied to the convolutional layers. The generator is comprised of a dense layer and a pair of strided transposed-convolutional layers (also known as deconvolutional layers) having 128 and 64 filters (stride length=2, kernel dimension=4) with associated Leaky Relu layers to map the input latent vector to a higher dimensional space. There is a final convolutional layer, having a single filter of kernel dimension=7 with a ‘tanh’ activation function to convert the feature-map to the desired shape of ECG cycle.

Training of the ensemble GAN: Separate mini-batches of real and fake data are used for training. The real and the fake samples are annotated as 1 and 0. The discriminator of each GAN aims to maximize the probability of correctly classifying an input as real or fake. The loss is expressed as Dₗₒₛₛ=log(D(x))+log(1−D(G(z))). These two terms are separately calculated on the mini-batches for real and generated fake data, providing a forward pass through the discriminator, and the gradients are calculated through a backward pass. For the generator, the loss term is Gₗₒₛₛ=log(D(G(z))). It tries to maximize log(D(G(z))), which is achieved by minimizing the term log(1−D(G(z))). The loss is calculated based on the classification output of the generated data as predicted by the discriminator.

In order to ensure the ECG cycles generated by the DCGAN are close to real ECG morphology, the Mean Squared Error (MSE) between the real and the generated data is added to the generator loss function of the DCGAN as a penalty term to be minimized. Hence the total generator loss of the DCGAN is as shown in equation (4) below.

$${G}_{{loss}_{t o t}}^{DCGAN} = {G}_{loss}^{DCGAN} + λ * \frac{1}{n} \sum {( {Y}_{i} - {\hat{Y}}_{i} )}^{2} \qquad ( 4 )$$

Y and Ŷ are the real and generated data, n is the batch size and the constant λ controls the weight of the penalty term. A small value is set as λ=0.05, so that newer samples are generated keeping the morphology similar to real ECG cycles.

Adam optimizer with a learning rate of 0.0002 is used for the LSTM-GAN and the DCGAN. The mini-batch size is set as 64. The model weights are initialized from a normal distribution with zero mean and standard deviation of 0.02. Label smoothing is applied to modify the hard labels for real data slightly more or less than 1 and slightly more than 0 for fake data, where the variation for each label is done randomly. Additionally, some noise is introduced in the labels by randomly flipping the labels of a small fraction of real and fake data in each mini-batch. Both techniques have a regularization effect to avoid over-fit. The composite network is trained end to end up to 500 epochs applying the same set of latent vectors to the generator modules of the two GANs in every mini-batch.

Generating the complete ECG waveform: Once the training is done, the generator of the LSTM-GAN and the DCGAN can generate a vector of R-R interval distances of length 50 and an ECG cycle of length 200 from an input latent vector. The complete ECG waveform is created by modifying the length of the generated ECG cycle according to the R-R interval distances using cubic spline interpolation and merging them on time axis. The signal is applied to a 4th order Butterworth bandpass filter, having cut-off frequencies of 0.5 Hz and 20 Hz to remove the noise components.

FIG. 3A and FIG. 3B illustrate ECG patterns of a normal subject and an AF patient, as known in the art. As seen in FIG. 3A and FIG. 3B, a normal ECG cycle has three major components, a P wave, a QRS complex containing an R peak, and the T wave. AF is a type of arrhythmia that affects the atrial activities of the heart. Firstly, the P waves are either absent or are replaced by fibrillatory waves. Secondly, the irregular Heart Rate Variability (HRV) due to AF causes large variation in successive R-R interval distances. These two clinical markers of AF are utilized in the present disclosure for synthesis of ECG signals.

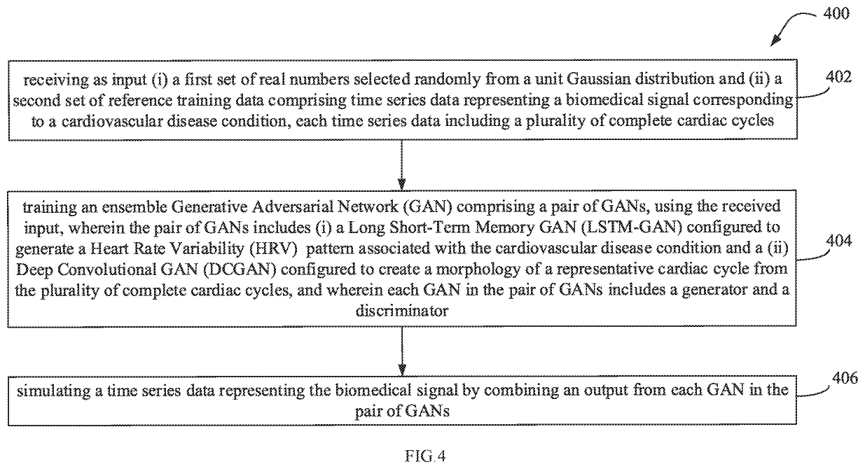

FIG. 4 illustrates an exemplary flow diagram of a computer implemented method 400 for simulation of cardiovascular disease specific biomedical signals using the ensemble GAN, in accordance with some embodiments of the present disclosure. In an embodiment, the system 100 includes one or more data storage devices or memory 102 operatively coupled to the one or more hardware processors 104 and is configured to store instructions configured for execution of steps of the method 400 by the one or more hardware processors 104. The steps of the method 400 will now be explained in detail with reference to the components of the system 100 of FIG. 1 and the exemplary architecture of FIG 2. Although process steps, method steps, techniques or the like may be described in a sequential order, such processes, methods and techniques may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

In an embodiment of the present disclosure, the one or more hardware processors 104, are configured to receive as input, at step 402, (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles. The reference training data has the signature of the cardiovascular disease condition.

In an embodiment, the one or more hardware processors 104, are configured to train, at step 404, the ensemble GAN (108 of FIG. 1) explained with reference to FIG. 2 above. In an embodiment, the step of training the ensemble GAN comprises, performing for each training epoch, the steps of: generating an R-R interval time series, by the generator of the LSTM-GAN, using the first set of real numbers by mapping the first set of real numbers to a time series and classifying the generated R-R interval time-series as belonging to the cardiovascular disease condition or not, by the discriminator of the LSTM-GAN based on R-R interval distances computed using the second set of reference training data; and generating the representative cardiac cycle specific to the cardiovascular disease condition, by the generator of the DCGAN, using the first set of real numbers and classifying the generated representative cardiac cycle as belonging to the cardiovascular disease condition or not, by the discriminator of the DCGAN based on the cardiac cycle computed using the second set of reference training data. In accordance with the present disclosure, the step of training culminates after a first predefined number of training epochs or when the generated R-R interval time series and the generated representative cardiac cycle is within a predefined threshold when compared with the second set of reference training data.

In an embodiment, the step 404 of training the ensemble GAN is made efficient by validating the output (simulated time series data) after every second predefined number of training epochs. The time series data representing the biomedical signal is simulated by combining an output from each GAN in the pair of GANs, repeatedly, after the second predefined number of training epochs, where in the second predefined number of training epochs is less than the first predefined number of training epochs. For instance, an internal periodic validation may be set after every 5 training epochs or every 10 training epochs and the step of training may be stopped if the simulated time series data is within the predefined threshold when compared with the second set of reference training data (saturation of classification result). This makes the model more reliable. Once the training is complete, the discriminator may be removed and only the generator may be retained.

Further, the one or more hardware processors 104, are configured to simulate, at step 406, a time series data representing the biomedical signal by combining an output from each GAN in the pair of GANs. In an embodiment, the step of simulating the time series data representing the biomedical signal comprises modifying length of the representative cardiac cycle generated by the DCGAN according to the R-R interval distances generated by the LSTMGAN using cubic spline interpolation and appending the representative cardiac cycles on a time axis.

### EXPERIMENTAL RESULTS

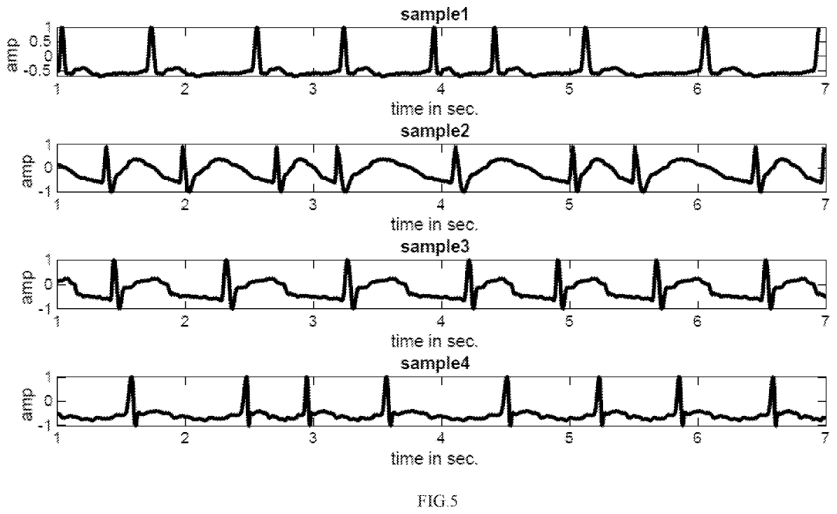

The architecture of the ensemble GAN of the present disclosure was implemented in Python™ 3.6 using TensorFlow 1.15 library. The training was performed on a computer with having Intel™ i7-7820X processor, 16 GB primary memory, and a GeForce™ GTX 1080 Ti graphics processing unit. FIG. 5 illustrates sample ECG waveforms generated, in accordance with some embodiments of the present disclosure. Irregular HRV pattern, the primary marker for AF is visible in all of them. Absence of P wave before the QRS complex is found in the first two samples, whereas the remaining samples show traces of fibrillatory waves.

The efficacy of the system and method of the present disclosure in data augmentation to mitigate the class imbalance problem of a dataset is quantitatively evaluated on the PhysioNet Challenge 2017 database. This annotated ECG database contains 5154 normal, 771 AF, and 2557 other types of abnormal recordings. The annotated database is converted to a highly imbalanced database for binary classification by merging all types of non-AF recordings under a single class. The re-labelled database contains 7711 non-AF and 771 AF recordings. For evaluating the impact of data augmentation in classification performance, two state-of-the-art supervised learning-based AF detectors were selected which were validated on the original database.

The first algorithm is a classical machine learning approach by Datta et. al. (Computing, vol. 44, pp. 1, 2017), that trains a series of cascaded binary AdaBoost classifiers using more than 150 hand-crafted features, related to ECG morphology and short-term HRV. The second algorithm by Zihlmann et. al.(Computing, vol. 44, pp. 1, 2017), proposes two separate deep learning classifiers based on Convolutional Neural Network Architecture (CNN) and CRNN [combination of convolutional neural network (CNN) and recurrent neural network (RNN)], taking the 2-D spectrogram of ECG as input. The algorithms are modified to binary classifiers for the experimental purpose. 80% of data from both classes is selected for training and the remaining portion for testing. The AF portion in the training set is used for data augmentation to balance the class ratio of AF to non-AF data. Subsequently, the AF classifiers are trained on the balanced training set and evaluated on the test set. Classification performance is reported in terms of sensitivity (Se) and specificity (Sp) of detecting AF as shown in equation (5) below.

$$S e = \frac{T P}{T P + F N} , Sp = \frac{T N}{T N + F P} \qquad ( 5 )$$

TP, TN, FP and FN indicate the true positive, true negative, false positive and false negative respectively.

Table 1 below shows the performance improvement achieved by the first machine learning based algorithm, when the training is done incorporating the ensemble GAN architecture for data augmentation. A classifier trained on a highly imbalanced database is expected to be biased towards the majority class. A significant number of AF recordings are misidentified when the classifier is trained on the original dataset, resulting in high specificity and low sensitivity. Synthetic Minority Oversampling Technique (SMOTE) [Chawla et al., in Journal of artificial intelligence research, vol 16, pp. 321-357, 2002] and Adaptive Synthetic (ADASYN) [He et al., in IEEE, 2008, PP. 1322-1328] are popularly used in machine learning for data augmentation. They simulate new data-points based on local information from the hand-crafted feature values computed from the AF-specific ECG signals. Although the sensitivity of AF improves due to them, there is a negative impact on specificity. The ensemble GAN, of the present disclosure generates newer ECG waveforms via learning the original class distribution. Hence, the features computed from the generated waveforms are found more effective than SMOTE and ADASYN, which significantly improves the classifier sensitivity without affecting the specificity.

TABLE 1
Quantitative analysis of the machine learning based AF
classifier by Datta et. al., applying various data augmentation
techniques to improve the class imbalance of the training
set in the PhysioNet Challenge database.

| Augmentation | | Augmentation | | Augmentation | | Augmentation | |
|---|---|---|---|---|---|---|---|
| (not used) | | (SMOTE) | | (ADASYN) | | (ensemble GAN) | |
| Se | Sp | Se | Sp | Se | Sp | Se | Sp |
| 0.81 | 0.96 | 0.84 | 0.93 | 0.86 | 0.93 | 0.91 | 0.96 |

Table 2 below shows that the ensemble GAN based augmentation has a similar impact on the CNN and CRNN based AF classifiers by Zihlmann et. al. Here, the ensemble GAN architecture of the present disclosure is compared with two different approaches. The first approach is a popular trick in deep learning, where the AF classifier is trained on the original imbalanced dataset by assigning 10 times higher class weight to the minority class, which pays more attention to that class. In spite of a significant improvement in sensitivity, it shows a negative impact on specificity. The second approach is the GAN architecture by Hatamian et al. in IEEE 2020, pp. 1264-1268, that generates spectrogram of ECG without reconstructing the time-series. Unlike the second approach, the ensemble GAN, of the present disclosure is designed based on the clinical biomarkers of AF, showing the capability to generate realistic ECG to improve the diversity of the training set. Thus, it has the optimum impact on classifier performance.

TABLE 2
Quantitative analysis of the deep learning-based AF classifiers
by Zihlmann et. al, applying various data augmentation techniques
to improve the class imbalance of the training set.

| | | | | | Augmenta- | | | |
|---|---|---|---|---|---|---|---|---|
| | | | | | tion (GAN | | Augmenta- | |
| | Augmentation | | Augmentation | | by Hatamian | | tion (ensem- | |
| | (not used) | | (class weight) | | et al.) | | ble GAN) | |
| | Se | Sp | Se | Sp | Se | Sp | Se | Sp |
| CNN | 0.79 | 0.99 | 0.89 | 0.91 | 0.83 | 0.98 | 0.92 | 0.99 |
| CRNN | 0.81 | 0.98 | 0.92 | 0.93 | 0.89 | 0.96 | 0.95 | 0.98 |

Thus, the present disclosure effectively combines two independent domain knowledges (i) physiological domain knowledge and (ii) deep learning to artificially simulate realistic cardiovascular disease specific biomedical signals. LSTM and CNN are the basic building blocks of LSTM-GAN and DCGAN respectively. Both LSTM and CNN are popular in deep learning. LSTMs are typically used for temporal analysis of a time-series to predict a next state, whereas, CNNs are mostly used for spatial analysis of images to extract its relevant features, which can be used for image classification, restoration and new image generation. Hence, CNN and LSTM are typically used in different domains. Biomedical signals such as ECG is a time-series, hence, to simulate it LSTM is an obvious choice for a person skilled in the art. However, it is found that LSTMs cannot always match ECG patterns. An ECG is complex in nature. Moreover, an abnormal ECG contains various anomalous patterns which is difficult to simulate using pure temporal analysis by an LSTM-GAN. Hence, DCGAN is introduced additionally on top, in the present disclosure which considers the morphology of an ECG cycle as an image to simulate a pattern base on spatial feature extraction by CNN. Thus, the ensemble GAN architecture including the LSTM-GAN and the DCGAN performs a detailed spatio-temporal modeling of biomedical signals such as the ECG signal, to regenerate more realistic artificial data and provides a technical advance to the ensemble GAN.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means, and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more hardware processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor implemented method comprising the steps of:
receiving as input, via one or more hardware processors, (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles;
training an ensemble Generative Adversarial Network (GAN) comprising a pair of GANs, via the one or more hardware processors, using the received input, wherein the pair of GANs includes (i) a Long Short-Term Memory GAN (LSTM-GAN) configured to generate a Heart Rate Variability (HRV) pattern associated with the cardiovascular disease condition and (ii) a Deep Convolutional GAN (DCGAN) configured to create a morphology of a representative cardiac cycle from the plurality of complete cardiac cycles, and wherein each GAN in the pair of GANs includes a generator and a discriminator; and
simulating, via the one or more hardware processors, a time series data representing the biomedical signal by combining an output from each GAN in the pair of GANs.

2. The processor implemented method of claim 1, wherein the step of training an ensemble GAN comprises performing, for each training epoch, the steps of:
generating an R-R interval time series, by the generator of the LSTM-GAN, using the first set of real numbers by mapping the first set of real numbers to a time series and classifying the generated R-R interval time-series as belonging to the cardiovascular disease condition or not, by the discriminator of the LSTM-GAN based on R-R interval distances computed using the second set of reference training data; and
generating the representative cardiac cycle specific to the cardiovascular disease condition, by the generator of the DCGAN, using the first set of real numbers and classifying the generated representative cardiac cycle as belonging to the cardiovascular disease condition or not, by the discriminator of the DCGAN based on the cardiac cycle computed using the second set of reference training data,
wherein the step of training culminates after a first predefined number of training epochs or when the generated R-R interval time series and the generated representative cardiac cycle is within a predefined threshold when compared with the second set of reference training data.

3. The processor implemented method of claim 2, further comprising periodically validating the step of training by simulating the time series data representing the biomedical signal by combining an output from each GAN in the pair of GANs, repeatedly, after every second predefined number of training epochs, where in the second predefined number of training epochs is less than the first predefined number of training epochs.

4. The processor implemented method of claim 3, wherein the step of simulating the time series data representing the biomedical signal comprises modifying length of the representative cardiac cycle generated by the DCGAN according to the R-R interval distances generated by the LSTMGAN using cubic spline interpolation and appending the representative cardiac cycles on a time axis.

5. A system comprising:
one or more hardware processors;
one or more communication interfaces;
one or more data storage devices operatively coupled to the one or more hardware processors and configured to store instructions configured for execution by the one or more hardware processors to:
receive as input (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles;
train an ensemble Generative Adversarial Network (GAN) comprising a pair of GANs, using the received input, wherein the pair of GANs includes (i) a Long Short-Term Memory GAN (LSTM-GAN) configured to generate a Heart Rate Variability (HRV) pattern associated with the cardiovascular disease condition and a (ii) Deep Convolutional GAN (DCGAN) configured to create a morphology of a representative cardiac cycle from the plurality of complete cardiac cycles, and wherein each GAN in the pair of GANs includes a generator and a discriminator; and
simulate a time series data representing the biomedical signal by combining an output from each GAN in the pair of GANs; and
the ensemble Generative Adversarial Network (GAN) comprising the pair of GANs.

6. The system of claim 5, wherein the one or more processors are configured to train the ensemble GAN by performing, for each training epoch, the steps of:
generating an R-R interval time series, by the generator of the LSTM-GAN, using the first set of real numbers by mapping the first set of real numbers to a time series and classifying the generated R-R interval time-series as belonging to the cardiovascular disease condition or not, by the discriminator of the LSTM-GAN based on R-R interval distances computed using the second set of reference training data; and
generating the representative cardiac cycle specific to the cardiovascular disease condition, by the generator of the DCGAN, using the first set of real numbers and classifying the generated representative cardiac cycle as belonging to the cardiovascular disease condition or not, by the discriminator of the DCGAN based on the cardiac cycle computed using the second set of reference training data,
wherein the step of training culminates after a first predefined number of training epochs or when the generated R-R interval time series and the generated representative cardiac cycle is within a predefined threshold when compared with the second set of reference training data.

7. The system of claim 6, wherein the one or more processors are configured to periodically validate the step of training by simulating the time series data representing the biomedical signal by combining an output from each GAN in the pair of GANs, repeatedly, after every second predefined number of training epochs, where in the second predefined number of training epochs is less than the first predefined number of training epochs.

8. The system of claim 7, wherein the one or more processors are configured to simulate the time series data representing the biomedical signal by modifying length of the representative cardiac cycle generated by the DCGAN according to the R-R interval distances generated by the LSTMGAN using cubic spline interpolation and appending the representative cardiac cycles on a time axis.

9. A computer program product comprising a non-transitory computer readable medium having a computer readable program embodied therein, wherein the computer readable program, when executed on a computing device, causes the computing device to:
receive as input, via one or more hardware processors, (i) a first set of real numbers selected randomly from a unit Gaussian distribution and (ii) a second set of reference training data comprising time series data representing a biomedical signal corresponding to a cardiovascular disease condition, each time series data including a plurality of complete cardiac cycles;
training an ensemble Generative Adversarial Network (GAN) comprising a pair of GANs, via the one or more hardware processors, using the received input, wherein the pair of GANs includes (i) a Long Short-Term Memory GAN (LSTM-GAN) configured to generate a Heart Rate Variability (HRV) pattern associated with the cardiovascular disease condition and (ii) a Deep Convolutional GAN (DCGAN) configured to create a morphology of a representative cardiac cycle from the plurality of complete cardiac cycles, and wherein each GAN in the pair of GANs includes a generator and a discriminator; and
simulating, via the one or more hardware processors, a time series data representing the biomedical signal by combining an output from each GAN in the pair of GANs.

10. The computer program product of claim 9, wherein the computer readable program further causes the computing device to perform the step of training an ensemble GAN by performing, for each training epoch, the steps of:
generating an R-R interval time series, by the generator of the LSTM-GAN, using the first set of real numbers by mapping the first set of real numbers to a time series and classifying the generated R-R interval time-series as belonging to the cardiovascular disease condition or not, by the discriminator of the LSTM-GAN based on R-R interval distances computed using the second set of reference training data; and
generating the representative cardiac cycle specific to the cardiovascular disease condition, by the generator of the DCGAN, using the first set of real numbers and classifying the generated representative cardiac cycle as belonging to the cardiovascular disease condition or not, by the discriminator of the DCGAN based on the cardiac cycle computed using the second set of reference training data,
wherein the step of training culminates after a first predefined number of training epochs or when the generated R-R interval time series and the generated representative cardiac cycle is within a predefined threshold when compared with the second set of reference training data.

11. The computer program product of claim 10, wherein the computer readable program further causes the computing device to periodically validate the step of training by simulating the time series data representing the biomedical signal by combining an output from each GAN in the pair of GANs, repeatedly, after every second predefined number of training epochs, wherein the second predefined number of training epochs is less than the first predefined number of training epochs.

12. The computer program product of claim 11, wherein the computer readable program further causes the computing device to simulate the time series data representing the biomedical signal by modifying length of the representative cardiac cycle generated by the DCGAN according to the R-R interval distances generated by the LSTMGAN using cubic spline interpolation and appending the representative cardiac cycles on a time axis.
